(19) United States
(12) Patent Application Publication
(10) Pub. No.: US 2025/0177341 A1
(43) Pub. Date: Jun. 5, 2025

(54) DIETARY TRIPROPIONIN SUPPLEMENTATION TO REDUCE ADIPOSITY AND IMPROVE GLUCOSE HOMEOSTASIS

(71) Applicant: Wisconsin Alumni Research Foundation, Madison, WI (US)

(72) Inventors: Federico Rey, Madison, WI (US); Evan Hutchison, Madison, WI (US); Chi-Liang Eric Yen, Madison, WI (US)

(73) Assignee: Wisconsin Alumni Research Foundation, Madison, WI (US)

(21) Appl. No.: 18/962,163

(22) Filed: Nov. 27, 2024

Related U.S. Application Data
(60) Provisional application No. 63/604,571, filed on Nov. 30, 2023.

(51) Int. Cl. A61K 31/225; A61K 9/00; A61K 31/365; A61P 3/04; A61P 3/08; A61P 5/50

(52) U.S. Cl. CPC A61K 31/225; A61K 9/0053; A61K 31/365; A61P 3/04; A61P 3/08; A61P 5/50

(57) ABSTRACT

Oral administration of tripropionin for reducing adiposity and/or improving glucose homeostasis. The oral administration of tripropionin can reduce body weight, reduce fat mass, improve glucose tolerance, improve insulin sensitivity, and/or reduce respiratory quotient in a subject.

### CROSS-REFERENCE TO RELATED APPLICATIONS

Priority is claimed to U.S. Provisional Application 63/604,571, filed Nov. 30, 2023, which is incorporated herein by reference in its entirety.

### STATEMENT REGARDING FEDERALLY SPONSORED RESEARCH

This invention was made with government support under HL148577 awarded by the National Institutes of Health. The government has certain rights in the invention.

### FIELD OF THE INVENTION

The invention is directed to the oral administration of tripropionin for purposes such as reducing adiposity and/or improving glucose homeostasis.

### BACKGROUND

Effective treatments for obesity, hyperglycemia (e.g., prediabetes, diabetes), insulin resistance, and related conditions (e.g. metabolic syndrome, Alzheimer's disease, atherosclerosis, fatty liver disease) are lacking. Current treatments have limited effectiveness, are associated with serious side effects, and are expensive.

Some studies have suggested that endogenously produced short-chain fatty acids in the gut might be associated with a number of beneficial health outcomes, including improved glucose homeostasis and reduced obesity. Such short-chain fatty acids—primarily acetate, propionate, and butyrate—are produced in the intestine through fermentation of carbohydrates and amino acids. However, the effects of short-chain fatty acids on glucose homeostasis and obesity are not clear. One study showed that oral administration of propionate in mice and humans leads to hyperglycemia, hyperinsulinemia, and weight gain (Tirosh et al. 2019). Another study found that propionate levels are higher in obese individuals than non-obese individuals (Fernandes et al. 2014). Another study found that fecal propionate levels were negatively associated with insulin sensitivity (Müller et al. 2019).

Effective treatments for obesity, hyperglycemia, insulin resistance, and related conditions are needed.

### SUMMARY OF THE INVENTION

One aspect of the invention is directed to methods of reducing adiposity and/or improving glucose homeostasis in a subject. In some versions, the methods comprise orally administering an effective amount of isolated tripropionin to the subject.

In some versions, the tripropionin is administered in an amount effective to: reduce body weight in the subject; reduce fat mass in the subject; improve glucose tolerance in the subject; improve insulin sensitivity in the subject; and/or reduce respiratory quotient of the subject.

In some versions, the tripropionin is administered in an amount effective to reduce body weight in the subject.

In some versions, the tripropionin is administered in an amount effective to reduce fat mass in the subject.

In some versions, the tripropionin is administered in an amount effective to improve glucose tolerance in the subject.

In some versions, the tripropionin is administered in an amount effective to improve insulin sensitivity in the subject.

In some versions, the tripropionin is administered in an amount effective to reduce respiratory quotient of the subject.

In some versions, the subject, when the administering initially commences, has a body mass index greater than 25.

In some versions, the subject is a male who, when the administering initially commences, has a body fat percentage greater than 15%, or the subject is a female who, when the administering initially commences, has a body fat percentage greater than 25%.

In some versions, the subject, when the administering initially commences, has a fasting glucose greater than 100 mg/dL.

In some versions, the subject, when the administering initially commences, has a condition selected from the group consisting of obesity, hyperglycemia, prediabetes, diabetes (e.g., type 1 diabetes type 2 diabetes, gestational diabetes, and maturity-onset diabetes of the young), insulin resistance, metabolic syndrome, Alzheimer's disease, atherosclerosis, and fatty liver disease. In some versions, the tripropionin is administered in an amount effective to treat the condition.

In some versions, the methods further comprise administering a lipase inhibitor to the subject.

In some versions, the tripropionin is administered in a composition comprising the tripropionin and a carrier.

In some versions, the composition is a solid or semisolid.

In some versions, the composition is in the form a pill. In some versions, the pill comprises the tripropionin in an amount of at least 25 mg.

In some versions, the composition is a liquid.

In some versions, the composition comprises the tripropionin in an amount of at least 1% w/w. In some versions, the composition comprises the tripropionin in an amount of at least 5% w/w.

In some versions, the composition has a caloric density no greater than 0.15 kcal/g.

In some versions, the composition has a caloric density of at least 0.4 kcal/g.

In some versions, at least 5% of total calories in the composition is protein.

In some versions, at least 5% of total calories in the composition is digestible carbohydrate.

In some versions, at least 5% of total calories in the composition is fat.

In some versions, the composition further comprises a lipase inhibitor. In some versions, the lipase inhibitor comprises orlistat.

Another aspect of the invention is directed compositions comprising isolated tripropionin.

Another aspect of the invention is directed to a composition of the invention for use in a method of reducing adiposity and/or improving glucose homeostasis in a subject. The composition can be for orally administering an effective amount of isolated tripropionin to the subject for reducing adiposity and/or improving glucose homeostasis in the subject. Any consideration pertaining to the methods and/or compositions outlined above or otherwise outlined herein can apply to such uses.

Another aspect of the invention is directed to the use of a compound of the invention in the manufacture of a medicament for reducing adiposity and/or improving glucose homeostasis in a subject. Any consideration pertaining to the methods and/or compositions outlined above or otherwise outlined herein can apply to such uses.

The objects and advantages of the invention will appear more fully from the following detailed description of the preferred embodiment of the invention made in conjunction with the accompanying drawings.

### BRIEF DESCRIPTION OF THE DRAWINGS

FIGS. 1A-1J. Effect of tripropionin on metabolic function. (FIG. 1A) Structure of tripropionin and the experimental design. (FIG. 1B) Bodyweight gain and (FIG. 1C) food consumption during the first 36 days on HFD. (FIG. 1D) Oral glucose tolerance test and (FIG. 1E) areas under the curve for the glucose tolerance test. (FIG. 1F) Insulin tolerance test and (FIG. 1G) areas under the curve for the insulin tolerance test. (FIG. 1H) Body weight, (FIG. 1I) fat mass, and (FIG. 1J) lean mass of mice in the final two weeks of the experiment. Student's T-test (P-value<0.05*, 0.005**, 0.00005****). All comparisons were done between diets at each timepoint. GL: High-fat diet (HFD)+5.3% w/w glycerol. TP: HFD+5.3% w/w tripropionin. In FIGS. 1B, 1C, 1E, 1G, 1H, 1I, and 1J, solid curves represent HFD+5.3% w/w glycerol, and dashed curves represent HFD+5.3% w/w tripropionin.

FIGS. 2A-2F. Effect of tripropionin on obese mice. (FIG. 2A) Experimental design. Measurement of body weight (FIG. 2B) and fat (FIG. 2C) and lean (FIG. 2D) mass by NMR. All mice were fed HFD prior to the switch to the experimental diets. (FIG. 2E) Continuous 48-hour monitoring of respiratory quotient. (FIG. 2F) Average food intake. Student's T-test (P-value <0.05*, 0.005**). All comparisons were done between diets at each timepoint. GL and Gly: HFD+5.3% w/w glycerol. TP and Tri: HFD+5.3% w/w tripropionin. In FIGS. 2B, 2C, and 2D, solid curves represent HFD+5.3% w/w glycerol, and dashed curves represent HFD+5.3% w/w tripropionin.

FIGS. 3A-3F. Effect of tripropionin on gnotobiotic mice colonized with a complex human microbial community. (FIG. 3A) Experimental design. (FIG. 3B) Difference in body weight relative to initial time point. (FIG. 3C) Oral glucose tolerance test and (FIG. 3D) areas under the curve for the oral glucose tolerance test. (FIG. 3E) Insulin tolerance test and (FIG. 3F) areas under the curve for the insulin tolerance test. Student's T-test (P-value <0.05*, 0.005**, 0.0005***). All comparisons were done between diets at each timepoint. GL: HFD+5.3% w/w glycerol. TP: HFD+5.3% w/w tripropionin. In FIGS. 3B, 3C, and 3E, solid curves represent HFD+5.3% w/w glycerol, and dashed curves represent HFD+5.3% w/w tripropionin.

### DETAILED DESCRIPTION OF THE INVENTION

On aspect of the invention is directed to the oral administration of isolated tripropionin to a subject. The administration can be effective, for example, to reduce adiposity and/or improve glycemic control in the subject. Accordingly, the isolated tripropionin can be administered in a therapeutically effective amount, such as an amount effective to reduce adiposity and/or improve glycemic control in the subject.

Tripropionin (2,3-di(propanoyloxy) propyl propanoate), also known as glyceryl tripropionate, is a triester of glycerol and propionic acid:

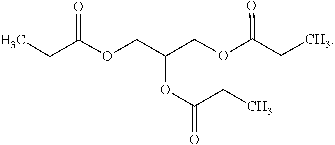 [SMILES: CCC(=O)OCC(COC(=O)CC)OC(=O)CC]

In pure form, it is a colorless, odorless, and tasteless liquid.

The term “isolated” used with reference to tripropionin refers to tripropionin that is synthetically produced or has been at least partially separated or purified away from at least one other component in which the tripropionin naturally occurs.

“Oral administration” or grammatical variants thereof (e.g., “orally administering”) refers to the administration such that the item administered is introduced via the mouth and enters at least into the stomach and/or downstream of the stomach in the gastrointestinal tract. Examples of oral administration can include ingestion and tube feeding, among other methods.

“Adiposity” as used herein refers to an amount of body fat in a subject. Adiposity is typically reported as the amount of fat mass (e.g., absolute mass of body fat or mass percent of body fat) in a subject. A number of methods of determining an amount of fat mass are known in the art and include the caliper method, bioelectrical impedance analysis, hydrostatic weighing, air displacement plethysmograph (e.g., “Bod Pod” technology), and dual energy x-ray absorptiometry (DEXA), among other methods. Adiposity can be indirectly reflected in a subject's body weight, waste circumference, waist:hip ratio, and/or body mass index (BMI). BMI is defined as kg/m2, where kg is a subject's weight in kilograms and m2 is the subject's height in meters squared.

“Reducing adiposity” and grammatical variants thereof refers to reducing the amount of body fat in a subject. A reduction in adiposity can be directly measured as a reduced amount of fat mass (e.g., absolute mass of body fat or mass percent of body fat) in the subject and/or indirectly reflected in a reduction in the subject's body weight, waste circumference, waist:hip ratio, and/or BMI.

Accordingly, in some versions of the invention, the tripropionin is administered in an amount effective to reduce fat mass in the subject. In some versions of the invention, the tripropionin is administered in an amount effective to reduce body weight in the subject. In some versions of the invention, the tripropionin is administered in an amount effective to reduce waste circumference in the subject. In some versions of the invention, the tripropionin is administered in an amount effective to reduce waist:hip ratio in the subject. In some versions of the invention, the tripropionin is administered in an amount effective to reduce BMI in the subject.

In some versions of the invention, the tripropionin is administered in an amount effective to reduce respiratory quotient of the subject. The respiratory quotient (RQ or respiratory coefficient) is a dimensionless number used in calculations of basal metabolic rate (BMR) when estimated from carbon dioxide production. It is calculated from the ratio of carbon dioxide produced by the body to oxygen consumed by the body. Such measurements, like measurements of oxygen uptake, are forms of indirect calorimetry. It is measured using a respirometer. The respiratory quotient value indicates which macronutrients are being metabolized, as different energy pathways are used for fats, carbohydrates, and proteins (Widmaier et al. 2016). If metabolism consists solely of lipids, the respiratory quotient is approximately 0.7, for proteins it is approximately 0.8, and for carbohydrates it is 1.0. Thus, a lower respiratory quotient can reflect a higher metabolism of fats. The respiratory quotient (RQ) is the ratio: RQ=CO₂ eliminated/O₂ consumed, where the term “eliminated” refers to carbon dioxide (CO₂) removed from the body. In this calculation, the CO₂ and O₂ must be given in the same units, and in quantities proportional to the number of molecules. Acceptable inputs would be moles or volumes of gas at standard temperature and pressure.

“Glucose homeostasis” and “glycemic control” are used interchangeably herein and refer to the ability to control blood glucose levels. An “improvement” in glucose homeostasis or glycemic control refers to an increased ability to control blood glucose levels. Three exemplary aspects of glucose homeostasis include glucose tolerance, insulin sensitivity, and fasting glucose levels.

In some versions of the invention, the tripropionin is administered in an amount effective to improve glucose tolerance in the subject. Glucose tolerance is the ability to dispose of a glucose load in the blood, such as after a meal. Glucose tolerance can be measured with a glucose tolerance test (GTT). A glucose tolerance test is performed by administering glucose to a subject and measuring blood glucose levels over time. A commonly performed version of a glucose tolerance test is an oral glucose tolerance test (OGTT), in which the glucose is administered by mouth. Glucose tolerance as determined in a glucose tolerance test is typically characterized by providing the area under a curve of blood glucose versus time. A lower area under the curve is understood to reflect higher glucose tolerance. A higher area under the curve is understood to reflect lower glucose tolerance. For the purposes herein, a reduced area under a curve of blood glucose versus time in a GTT is considered to constitute an improvement in glucose tolerance and an improvement in glucose homeostasis.

In some versions of the invention, the tripropionin is administered in an amount effective to improve insulin sensitivity in the subject. Insulin sensitivity is the sensitivity of the body to the effects of insulin, e.g., signaling for the disposal of glucose from the blood into peripheral tissues. Insulin sensitivity can be measured with an insulin tolerance test (ITT). An insulin tolerance test is performed by administering insulin to a subject and measuring blood glucose levels over time. Insulin sensitivity as determined in a glucose tolerance test is typically characterized by providing the area under a curve of blood glucose versus time. A lower area under the curve is understood to reflect higher insulin sensitivity. A higher area under the curve is understood to reflect lower glucose insulin sensitivity. For the purposes herein, a reduced area under a curve of blood glucose versus time in an ITT is considered to constitute an improvement in insulin sensitivity and an improvement in glucose homeostasis.

In some versions of the invention, the tripropionin is administered in an amount effective to reduce fasting glucose levels in the subject. For the purposes herein, a reduction in fasting glucose levels is considered to constitute an improvement in fasting glucose levels and an improvement in glucose homeostasis.

In some versions of the invention, the subject is an animal. In some versions of the invention, the subject is a mammal. In some versions of the invention, the subject is a human. In some versions of the invention, the subject is a companion animal. “Companion animal” as used herein refers to a domesticated or domestic-bread non-human animal. Exemplary companion animals include canines and felines. In some versions of the invention, the subject is a production animal. “Production animal” as used herein refers to a non-human animal used for meat, fiber, milk, or other products. Exemplary production animals include bovines, avians, and porcines. In some versions of the invention, the animal is a research animal. “Research animal” as used herein refers to animals bred for research use.

A reduction in adiposity with the administration of tripropionin can be particularly beneficial for a number of subjects, including those who are overweight or obese.

In some versions, the subject, when the administration initially commences, has a body fat percentage greater than 15%, such as greater than 16%, greater than 17%, greater than 18%, greater than 19%, greater than 20%, greater than 21%, greater than 22%, greater than 23%, greater than 24%, greater than 25%, greater than 26%, greater than 27%, greater than 28%, greater than 29%, greater than 30%, greater than 31%, greater than 32%, greater than 33%, greater than 34%, greater than 35%, greater than 36%, greater than 37%, greater than 38%, greater than 39%, or greater than 40%.

In some versions, the subject is a male and, when the administration initially commences, has a body fat percentage greater than 15%, such as greater than 16%, greater than 17%, greater than 18%, greater than 19%, greater than 20%, greater than 21%, greater than 22%, greater than 23%, greater than 24%, greater than 25%, greater than 26%, greater than 27%, greater than 28%, greater than 29%, greater than 30%, greater than 31%, greater than 32%, greater than 33%, greater than 34%, greater than 35%, greater than 36%, greater than 37%, greater than 38%, greater than 39%, or greater than 40%.

In some versions, the subject is a female and, when the administration initially commences, has a body fat percentage greater than 20%, such as greater than 21%, greater than 22%, greater than 23%, greater than 24%, greater than 25%, greater than 26%, greater than 27%, greater than 28%, greater than 29%, greater than 30%, greater than 31%, greater than 32%, greater than 33%, greater than 34%, greater than 35%, greater than 36%, greater than 37%, greater than 38%, greater than 39%, or greater than 40%.

In some versions, the subject, when the administration initially commences, has a body mass index greater than 25, such as greater than 26, greater than 27, greater than 28, greater than 29, greater than 30, greater than 31, greater than 32, greater than 33, greater than 34, greater than 35, greater than 36, greater than 37, greater than 38, greater than 39, or greater than 40.

An improvement in glycemic control with the administration of tripropionin can be particularly beneficial for a number of subjects, including those who have defects in glycemic control and/or are hyperglycemic (from e.g., prediabetes, diabetes, and/or insulin resistance).

In some versions, the subject, when the administration initially commences, has a fasting glucose greater than 100 mg/dL, greater than 105 mg/dL, greater than 110 mg/dL, greater than 115 mg/dL, greater than 120 mg/dL, greater than 125 mg/dL, greater than 130 mg/dL, greater than 135 mg/dL, greater than 140 mg/dL, greater than 145 mg/dL, greater than 150 mg/dL, greater than 160 mg/dL, greater than 170 mg/dL, greater than 180 mg/dL, greater than 190 mg/dL, greater than 200 mg/dL, greater than 210 mg/dL, greater than 220 mg/dL, greater than 230 mg/dL, greater than 240 mg/dL, greater than 250 mg/dL, greater than 260 mg/dL, greater than 270 mg/dL, greater than 280 mg/dL, greater than 290 mg/dL, greater than 300 mg/dL, greater than 350 mg/dL, greater than 400 mg/dL, greater than 450 mg/dL, greater than 500 mg/dL, greater than 550 mg/dL, or greater than 600 mg/dL.

In some versions, the tripropionin is administered in an amount effective to treat a condition. In various versions of the invention, the condition can comprise any one or more of obesity, hyperglycemia, prediabetes, diabetes, insulin resistance, metabolic syndrome, Alzheimer's disease, atherosclerosis, and fatty liver disease. “Treat” in this context refers to the any amelioration of the condition and any symptom associated therewith. Exemplary forms of diabetes that can be treated with the methods of the invention include type 1 diabetes type 2 diabetes, gestational diabetes, and maturity-onset diabetes of the young. Metabolic syndrome is a cluster of conditions that include hyperglycemia and excess body fat and thus can be treated with the methods of the invention. Alzheimer's disease (de la Monte S M. Insulin resistance and Alzheimer's disease. BMB Rep. 2009 Aug. 31; 42 (8): 475-81), atherosclerosis (Bornfeldt K E, Tabas I. Insulin resistance, hyperglycemia, and atherosclerosis. Cell Metab. 2011 Nov. 2; 14(5):575-85), and fatty liver disease (Gaggini M, Morelli M, Buzzigoli E, DeFronzo R A, Bugianesi E, Gastaldelli A. Non-alcoholic fatty liver disease (NAFLD) and its connection with insulin resistance, dyslipidemia, atherosclerosis and coronary heart disease. Nutrients. 2013 May 10; 5 (5): 1544-60) are all conditions associated with hyperglycemia and/or insulin resistance and thus can be treated with the methods of the invention.

In some versions of the invention, the subject to which the tripropionin is administered is a subject that has any one or more of the following conditions: obesity, hyperglycemia, prediabetes, diabetes (e.g., type 1 diabetes type 2 diabetes, gestational diabetes, and maturity-onset diabetes of the young), insulin resistance, metabolic syndrome, Alzheimer's disease, atherosclerosis, and fatty liver disease. In some versions, the subject to which the tripropionin is administered has, when the administering initially commences, any one or more of the following conditions: obesity, hyperglycemia, prediabetes, diabetes (e.g., type 1 diabetes type 2 diabetes, gestational diabetes, and maturity-onset diabetes of the young), insulin resistance, metabolic syndrome, Alzheimer's disease, atherosclerosis, and fatty liver disease.

The tripropionin can be administered in the form of a composition comprising the tripropionin. In general, the compositions can comprise the tripropionin in an amount of at least 0.01% w/w, at least 0.05% w/w, at least 0.1% w/w, at least 0.5% w/w, at least 1% w/w, at least 5% w/w, at least 10% w/w, at least 15% w/w, at least 20% w/w, at least 25% w/w, at least 30% w/w, at least 35% w/w, at least 40% w/w, at least 45% w/w, at least 50% w/w, at least 55% w/w, at least 60% w/w, at least 65% w/w, at least 70% w/w, at least 75% w/w, at least 80% w/w, at least 85% w/w, at least 90% w/w, at least 95% w/w, or at least 99% w/w. The composition can be a liquid, a solid, a semisolid, or a mixed-phase substance.

In some versions, the composition is in the form of a pill. As used herein, the term “pill” refers to a small solid, semi-solid, or solid-coated mass sized having a determinate unit dose of tripropionin and capable of being swallowed whole or chewed and swallowed. Examples of pills include capsules tablets, and caplets. Capsules typically include an outer shell, often made of gelatin, and contain powder, miniature pellets, or liquid inside. Types of capsules include hard-shelled capsules and soft gels, the latter of which typically have a soft gelatin shell. Tablets are pills composed of an active agent (tripropionin in the present case), diluents, binders, and/or granulating agents, among other options agents, that are compressed into a solid mass. Caplets are typically smooth-coated tablets.

In some versions, the pill has a weight from 50 mg to 1,000 mg, such as from 100 mg to 500 mg.

In some versions, the pill comprises the tripropionin in an amount of at least 1 mg, at least 10 mg, at least 20 mg, at least 30 mg, at least 40 mg, at least 50 mg, at least 60 mg, at least 70 mg, at least 80 mg, at least 90 mg, at least 100 mg, at least 110 mg, at least 120 mg, at least 130 mg, at least 140 mg, at least 150 mg, at least 160 mg, at least 170 mg, at least 180 mg, at least 190 mg, at least 200 mg, at least 210 mg, at least 220 mg, at least 230 mg, at least 240 mg, at least 250 mg, at least 260 mg, at least 270 mg, at least 280 mg, at least 290 mg, or at least 300 mg.

In some versions, the pill comprises the tripropionin in an amount of at least 0.01% w/w, at least 0.05% w/w, at least 0.1% w/w, at least 0.5% w/w, at least 1% w/w, at least 5% w/w, at least 10% w/w, at least 15% w/w, at least 20% w/w, at least 25% w/w, at least 30% w/w, at least 35% w/w, at least 40% w/w, at least 45% w/w, at least 50% w/w, at least 55% w/w, at least 60% w/w, at least 65% w/w, at least 70% w/w, at least 75% w/w, at least 80% w/w, at least 85% w/w, at least 90% w/w, at least 95% w/w, or at least 99% w/w.

In some versions, the composition is devoid of protein or comprises protein in an amount less than 95% w/w, less than 90% w/w, less than 85% w/w, less than 80% w/w, less than 75% w/w, less than 70% w/w, less than 65% w/w, less than 60% w/w, less than 55% w/w, less than 50% w/w, less than 45% w/w, less than 40% w/w, less than 35% w/w, less than 30% w/w, less than 25% w/w, less than 20% w/w, less than 15% w/w, less than 10% w/w, less than 5% w/w, or less than 1% w/w.

In some versions, the composition is devoid of digestible carbohydrate or comprises digestible carbohydrate in an amount less than 95% w/w, less than 90% w/w, less than 85% w/w, less than 80% w/w, less than 75% w/w, less than 70% w/w, less than 65% w/w, less than 60% w/w, less than 55% w/w, less than 50% w/w, less than 45% w/w, less than 40% w/w, less than 35% w/w, less than 30% w/w, less than 25% w/w, less than 20% w/w, less than 15% w/w, less than 10% w/w, less than 5% w/w, or less than 1% w/w. “Digestible” in this context refers to being digestible to humans. Digestible carbohydrates include starch, starch-based products, sucrose, lactose, glucose, some sugar alcohols, and unusual (and fairly rare) α-linked glucans, which provide caloric energy. Digestible carbohydrates are contrasted with non-digestible carbohydrates such as certain high molecular weight polysaccharides, mainly from plant cell walls, and other forms of dietary fiber.

In some versions, the composition is devoid of non-propionate fat or comprises non-propionate fat in an amount less than 95% w/w, less than 90% w/w, less than 85% w/w, less than 80% w/w, less than 75% w/w, less than 70% w/w, less than 65% w/w, less than 60% w/w, less than 55% w/w, less than 50% w/w, less than 45% w/w, less than 40% w/w, less than 35% w/w, less than 30% w/w, less than 25% w/w, less than 20% w/w, less than 15% w/w, less than 10% w/w, less than 5% w/w, or less than 1% w/w. “Non-propionate fat” refers to lipid other than propionate.

In some versions, the tripropionate is administered as a nutritional additive, either as a food or nutraceutical supplement.

In some versions, the composition comprises protein in an amount of at least 0.01% w/w, at least 0.05% w/w, at least 0.1% w/w, at least 0.5% w/w, at least 1% w/w, at least 5% w/w, at least 10% w/w, at least 15% w/w, at least 20% w/w, at least 25% w/w, at least 30% w/w, at least 35% w/w, at least 40% w/w, at least 45% w/w, at least 50% w/w, at least 55% w/w, at least 60% w/w, at least 65% w/w, at least 70% w/w, at least 75% w/w, at least 80% w/w, at least 85% w/w, at least 90% w/w, at least 95% w/w, or at least 99% w/w.

In some versions, the composition comprises digestible carbohydrate in an amount of at least 0.01% w/w, at least 0.05% w/w, at least 0.1% w/w, at least 0.5% w/w, at least 1% w/w, at least 5% w/w, at least 10% w/w, at least 15% w/w, at least 20% w/w, at least 25% w/w, at least 30% w/w, at least 35% w/w, at least 40% w/w, at least 45% w/w, at least 50% w/w, at least 55% w/w, at least 60% w/w, at least 65% w/w, at least 70% w/w, at least 75% w/w, at least 80% w/w, at least 85% w/w, at least 90% w/w, at least 95% w/w, or at least 99% w/w.

In some versions, the composition comprises non-propionate fat in an amount of at least 0.01% w/w, at least 0.05% w/w, at least 0.1% w/w, at least 0.5% w/w, at least 1% w/w, at least 5% w/w, at least 10% w/w, at least 15% w/w, at least 20% w/w, at least 25% w/w, at least 30% w/w, at least 35% w/w, at least 40% w/w, at least 45% w/w, at least 50% w/w, at least 55% w/w, at least 60% w/w, at least 65% w/w, at least 70% w/w, at least 75% w/w, at least 80% w/w, at least 85% w/w, at least 90% w/w, at least 95% w/w, or at least 99% w/w.

In some versions, the composition has a caloric density of no greater than 0.001 kcal/g, no greater than 0.005 kcal/g, no greater than 0.01 kcal/g, no greater than 0.05 kcal/g, no greater than 0.1 kcal/g, no greater than 0.2 kcal/g, no greater than 0.3 kcal/g, no greater than 0.4 kcal/g, no greater than 0.5 kcal/g, no greater than 0.6 kcal/g, no greater than 0.7 kcal/g, no greater than 0.8 kcal/g, no greater than 0.9 kcal/g, no greater than 1 kcal/g, 1.1 kcal/g, no greater than 1.2 kcal/g, no greater than 1.3 kcal/g, no greater than 1.4 kcal/g, no greater than 1.5 kcal/g, no greater than 1.6 kcal/g, no greater than 1.7 kcal/g, no greater than 1.8 kcal/g, no greater than 1.9 kcal/g, no greater than 2 kcal/g, 3.1 kcal/g, no greater than 3.2 kcal/g, no greater than 3.3 kcal/g, no greater than 3.4 kcal/g, no greater than 3.5 kcal/g, no greater than 3.6 kcal/g, no greater than 3.7 kcal/g, no greater than 3.8 kcal/g, no greater than 3.9 kcal/g, no greater than 4 kcal/g, no greater than 4.5 kcal/g, no greater than 5 kcal/g, no greater than 5.5 kcal/g, no greater than 6 kcal/g, no greater than 6.5 kcal/g, no greater than 7 kcal/g, no greater than 7.5 kcal/g, no greater than 8 kcal/g, no greater than 8.5 kcal/g, no greater than 9 kcal/g, or no greater than 10 kcal/g.

In some versions, the composition has a caloric density of at least 0.001 kcal/g, at least 0.005 kcal/g, at least 0.01 kcal/g, at least 0.05 kcal/g, at least 0.1 kcal/g, at least 0.2 kcal/g, at least 0.3 kcal/g, at least 0.4 kcal/g, at least 0.5 kcal/g, at least 0.6 kcal/g, at least 0.7 kcal/g, at least 0.8 kcal/g, at least 0.9 kcal/g, at least 1 kcal/g, 1.1 kcal/g, at least 1.2 kcal/g, at least 1.3 kcal/g, at least 1.4 kcal/g, at least 1.5 kcal/g, at least 1.6 kcal/g, at least 1.7 kcal/g, at least 1.8 kcal/g, at least 1.9 kcal/g, at least 2 kcal/g, 3.1 kcal/g, at least 3.2 kcal/g, at least 3.3 kcal/g, at least 3.4 kcal/g, at least 3.5 kcal/g, at least 3.6 kcal/g, at least 3.7 kcal/g, at least 3.8 kcal/g, at least 3.9 kcal/g, at least 4 kcal/g, at least 4.5 kcal/g, at least 5 kcal/g, at least 5.5 kcal/g, at least 6 kcal/g, at least 6.5 kcal/g, at least 7 kcal/g, at least 7.5 kcal/g, at least 8 kcal/g, or at least 8.5 kcal/g.

In some versions, at least 0.01%, at least 0.05%, at least 0.1%, at least 0.5%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of the total calories in the composition is protein.

In some versions, at least 0.01%, at least 0.05%, at least 0.1%, at least 0.5%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of the total calories in the composition is digestible carbohydrate.

In some versions, at least 0.01%, at least 0.05%, at least 0.1%, at least 0.5%, at least 1%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% of the total calories in the composition is fat. Fat in this context includes all fat, including tripropionin.

The amount of the tripropionin to be administered will vary depending on the subject, stage of disease or disorder, age of the subject, general health of the subject, and various other parameters known and routinely taken into consideration by those of skill in the medical arts. As a general matter, a sufficient amount of tripropionin will be administered in order to make a detectable change in the subject. Examples of amounts include from 1 ng/kg/day to 20 g/kg/day body weight, such as from 50 μg/kg/day to 5 g/kg/day, from 1 mg/kg/day to 100 mg/kg/day, from 1 μg/kg/day to 1 g/kg/day, or from 1 mg/kg/day to 100 mg/kg/day per day (wherein the foregoing units refer to tripropionin mass/subject body weight mass/time window). Suitable compositions can be formulated accordingly. Those of skill in the art of dosing of biologically active agents will be able to develop particular dosing regimens for various subjects based on known and well understood parameters.

The effective amount of tripropionin can be administered for periods ranging from about 1 to 1000 days or longer, such as from 7 to 300 days or from 30 to 90 days. The effective amount of tripropionin may be continued beyond these periods for maintenance of beneficial responses in chronic diseases.

Exemplary compositions of the invention are pharmaceutical compositions, such as in the form of tablets, pills, capsules, caplets, multi-particulates (including granules, beads, pellets and micro-encapsulated particles), powders, elixirs, syrups, suspensions, and solutions. Pharmaceutical compositions will typically comprise a pharmaceutically acceptable diluent or carrier. Pharmaceutical compositions are preferably adapted for oral administration. Orally administrable compositions may be in solid or liquid form and may take the form of tablets, powders, suspensions, and syrups, among other things. Optionally, the compositions can comprise one or more flavoring and/or coloring agents. In general, compositions of the invention may comprise any substance that does not significantly interfere with the action of tripropionin on the subject.

Pharmaceutically acceptable carriers suitable for use in pharmaceutical compositions are well known in the art of pharmacy. The pharmaceutical compositions of the invention may contain 0.01-99% by weight of tripropionin. The pharmaceutical compositions of the invention are generally prepared in unit dosage form. Examples of unit dosages of tripropionin include from 0.1 mg to 2000 mg, such as 50 mg to 1000 mg. The excipients used in the preparation of these compositions are the excipients known in the art.

To formulate tripropionin into compositions such as tablets, capsules, powders, granules, tripropionin is preferably mixed with a binder, a disintegrating agent and/or a lubricant. If necessary, the resultant composition may be mixed with a diluent, a buffer, an infiltrating agent, a preservative and/or a flavor, using known methods. Examples of the binder include crystalline cellulose, cellulose derivatives, cornstarch, cyclodextrins, and gelatin. Examples of the disintegrating agent include cornstarch, potato starch, and sodium carboxymethylcellulose. Examples of the lubricant include talc and magnesium stearate. Further, additives, which have been conventionally used, such as lactose and mannitol, may also be used. Soft gels or hard capsules can comprise an encapsulating material such as gelatin, starch, modified starch, starch derivatives such as glucose, sucrose, lactose, and fructose. The encapsulating material may optionally contain cross-linking or polymerizing agents, stabilizers, antioxidants, light absorbing agents for protecting light-sensitive fills, preservatives, and the like. To formulate the tripropionin into suspensions, syrups, or elixirs, a pharmaceutically suitable solvent may be used. Included among these is the non-limiting example of water.

Tripropionin may also be administered in a nutritional composition. The nutritional composition may be a food or nutraceutical supplement. The food can be supplemented with an amount of tripropionin, such as an effective amount. The nutraceutical supplement can be provided in the form of a pharmaceutical composition as outlined above.

When the effective amount of tripropionin is administered in a food or nutritional composition, an exemplary dose can range from about 0.001 to about 10.0% w/w of the food or nutraceutical composition.

In general, the term “carrier” represents one or more components with which tripropionin may be mixed, be it a pharmaceutical carrier, foodstuff, nutritional supplement, or dietary aid. The materials described above may be considered carriers for the purposes of the invention. In certain embodiments of the invention, the carrier has little to no biological activity.

The amounts of tripropionin defined herein as being effective to carry out a specific effect can be an amount effective to carry out the specific effect over time in the subject to which the tripropionin administered, and/or can be an amount effective to carry out the specific effects in a test subject administered a given amount of tripropionin relative to a control not administered the given amount of the tripropionin.

Some versions of the invention further comprise administering a lipase inhibitor to the subject. The lipase inhibitor can be included and administered in the same composition as the tripropionin, or can be administered separately, such as in a separate composition. Any composition, method of administration described herein for tripropionin can be employed for the lipase inhibitor. In some versions, the tripropionin and the lipase inhibitor are administered separately in a dosing regimen over a period of time, wherein the period of time of the dosing regimen for the tripropionin and the period of time of the dosing regimen for the lipase inhibitor overlap. In some cases, the tripropionin is administered at least twice within a period of time, and the lipase inhibitor is administered within the period of time. In some cases, the lipase inhibitor is administered at least twice within a period of time, and the tripropionin is administered within the period of time. In various versions, the period of time in any of such cases can be less than 1 hour, less than 2 hours, less than 12 hours, less than 1 day, less than 3 days, less than 1 week, less than 2 weeks, less than 3 weeks, less than 4 weeks, less than 5 weeks or any other suitable period of time.

Exemplary suitable lipase inhibitors include orlistat (CAS Number 96829-58-2), cetilistat (CAS Number 282526-98-1), lalistat 1, lalistat 2, RHC 80267, GSK 264220A, fucoxanthin, fucoxanthinol, hesperidin, a proanthocyanidin, luteolin, a flavan 3-ol monomer such as catechin and epicatechin, lipstatin, valilactone, percyquinin, panclicin (A-E), ebelactone (A and B), vibralactone, esterastin, (E)-4-amino styryl acetate, ε-polylysine, and caulerpenyne, among others. The lipase inhibitor can inhibit any type of lipase. In various versions, the lipase inhibitor is a pancreatic lipase inhibitor, a gastric lipase inhibitor, a carboxylester lipase inhibitor, a lysosomal acid lipase inhibitor, a diacylglycerol lipase inhibitor, an endothelial lipase, and/or a lipoprotein lipase inhibitor.

The elements and method steps described herein can be used in any combination whether explicitly described or not.

All combinations of method steps as used herein can be performed in any order, unless otherwise specified or clearly implied to the contrary by the context in which the referenced combination is made.

As used herein, the singular forms “a,” “an,” and “the” include plural referents unless the content clearly dictates otherwise.

Numerical ranges as used herein are intended to include every number and subset of numbers contained within that range, whether specifically disclosed or not. Further, these numerical ranges should be construed as providing support for a claim directed to any number or subset of numbers in that range. For example, a disclosure of from 1 to 10 should be construed as supporting a range of from 2 to 8, from 3 to 7, from 5 to 6, from 1 to 9, from 3.6 to 4.6, from 3.5 to 9.9, and so forth.

All patents, patent publications, and peer-reviewed publications (i.e., “references”) cited herein are expressly incorporated by reference to the same extent as if each individual reference were specifically and individually indicated as being incorporated by reference. In case of conflict between the present disclosure and the incorporated references, the present disclosure controls.

It is understood that the invention is not confined to the particular construction and arrangement of parts herein illustrated and described, but embraces such modified forms thereof as come within the scope of the claims.

### EXAMPLES

### Example 1

### Summary

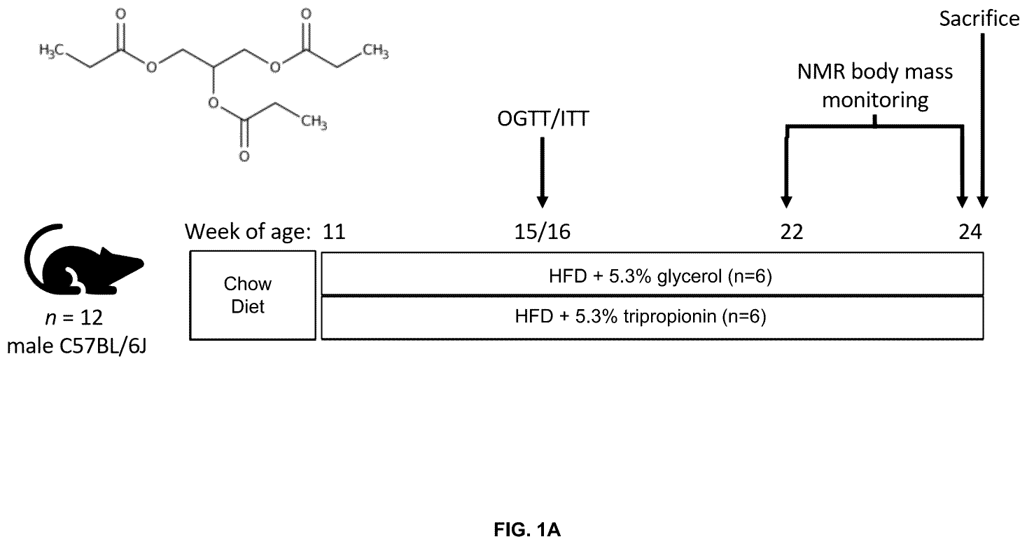

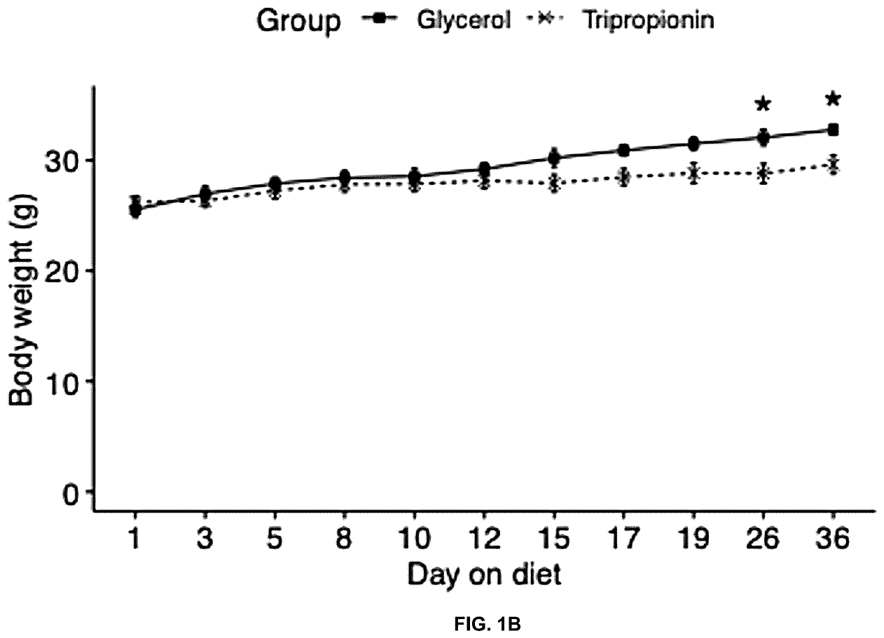

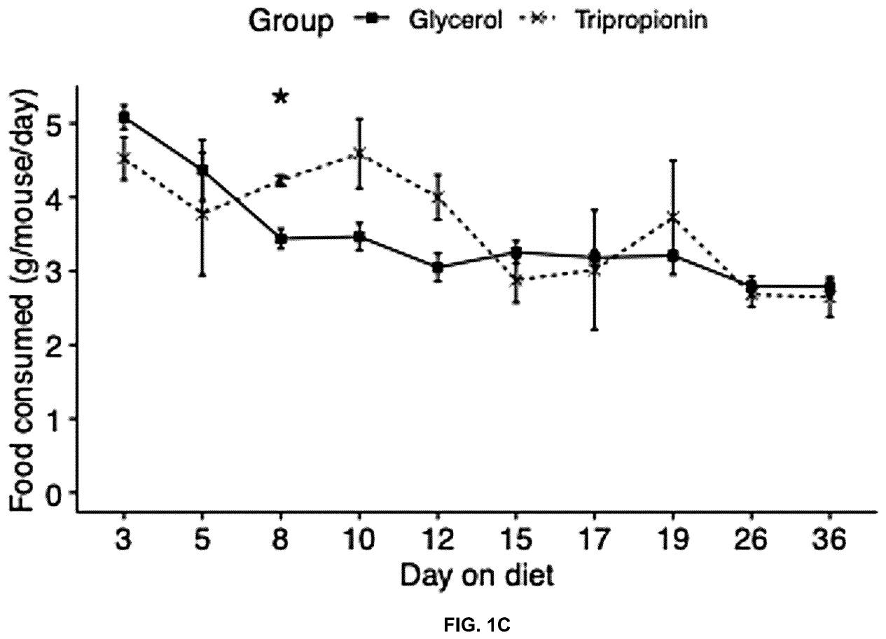

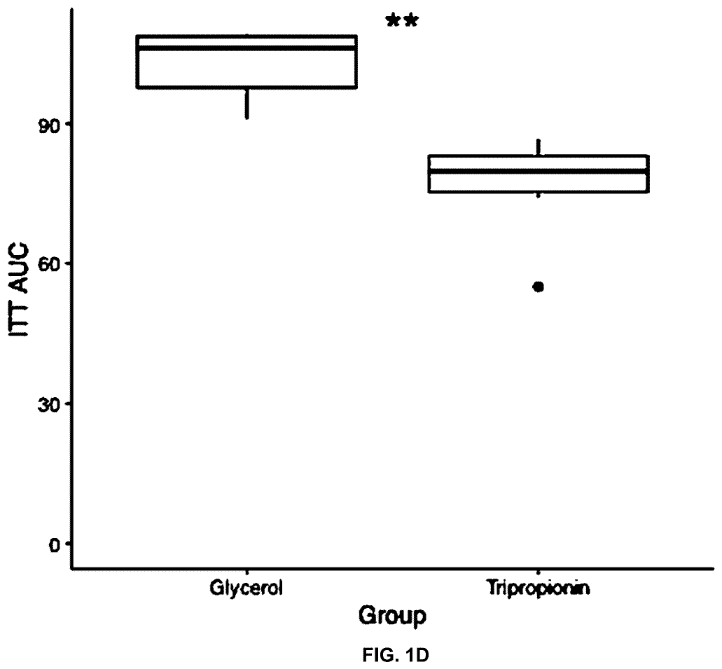

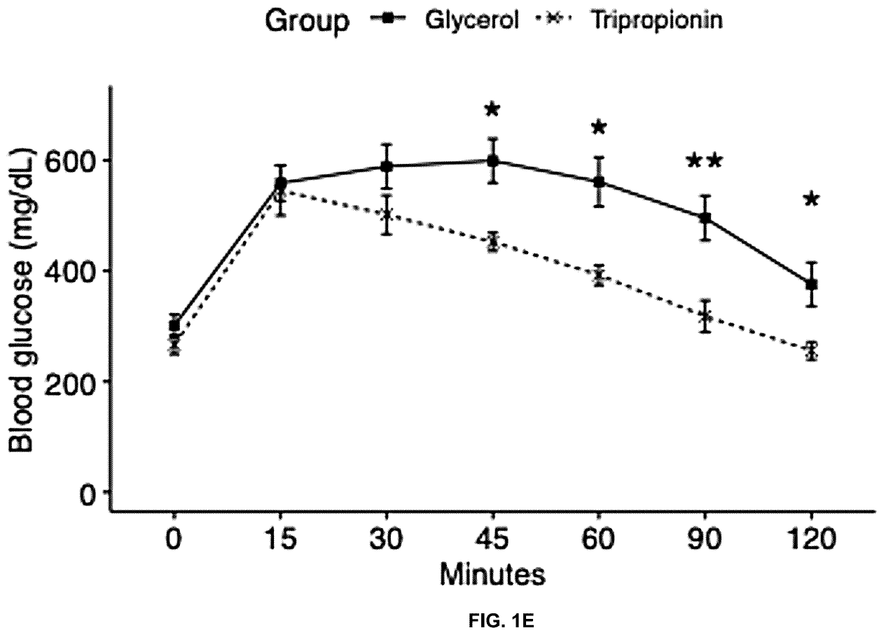

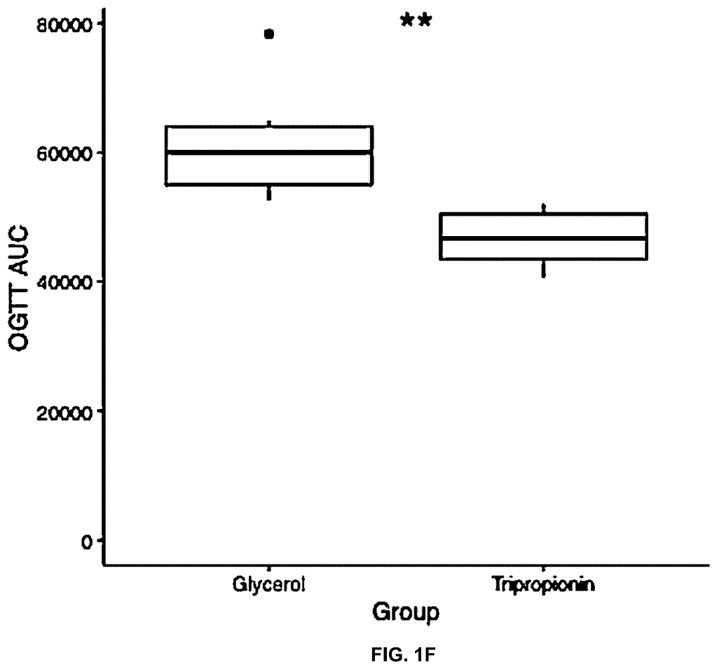

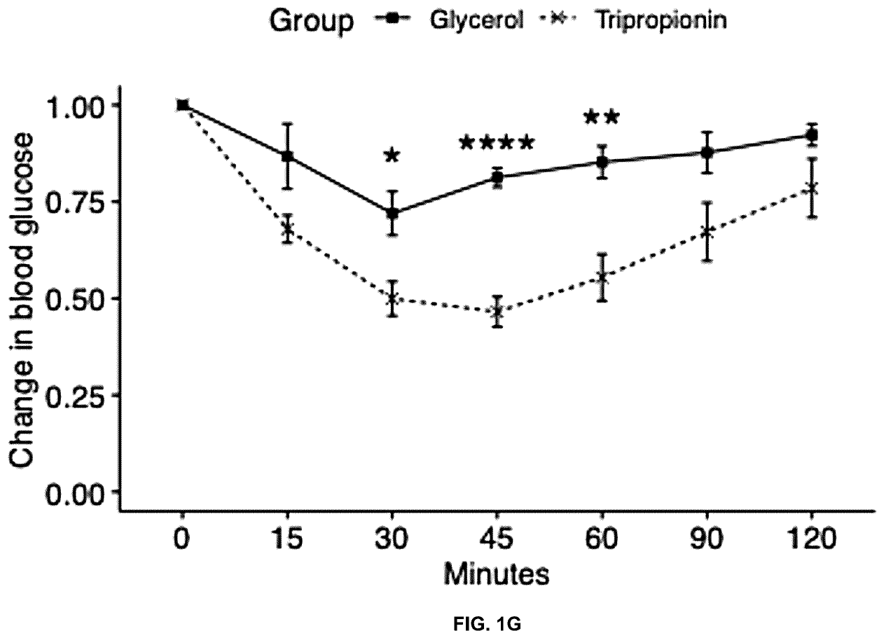

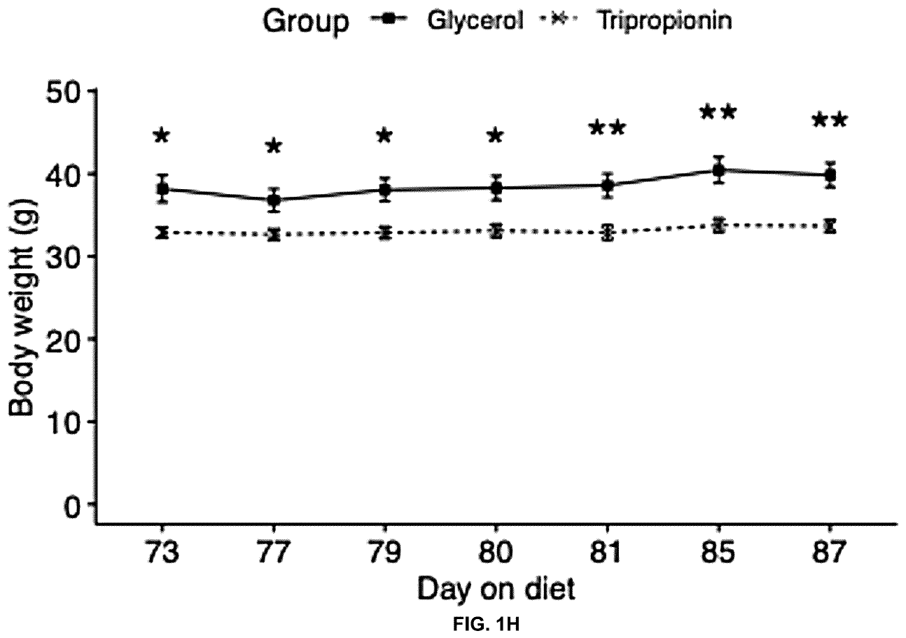

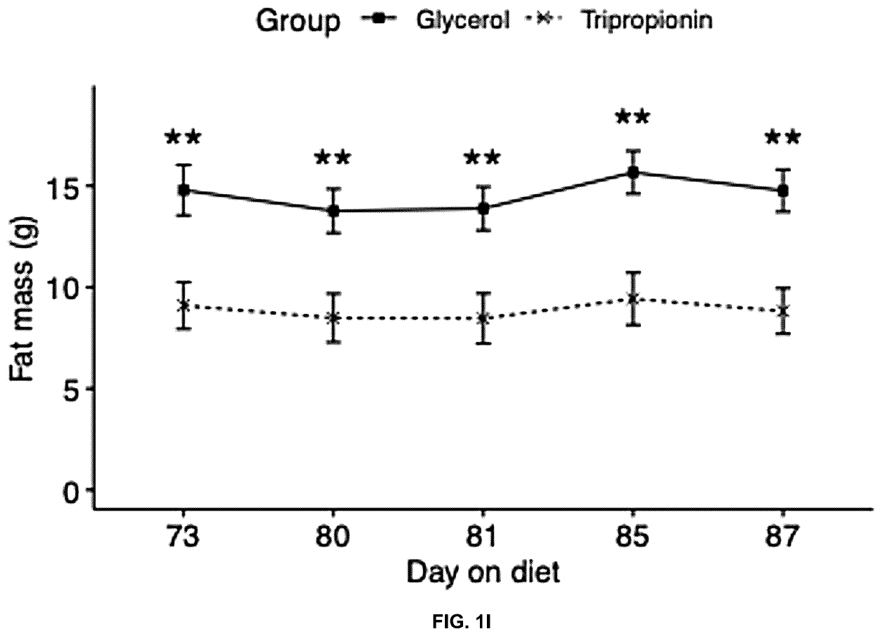

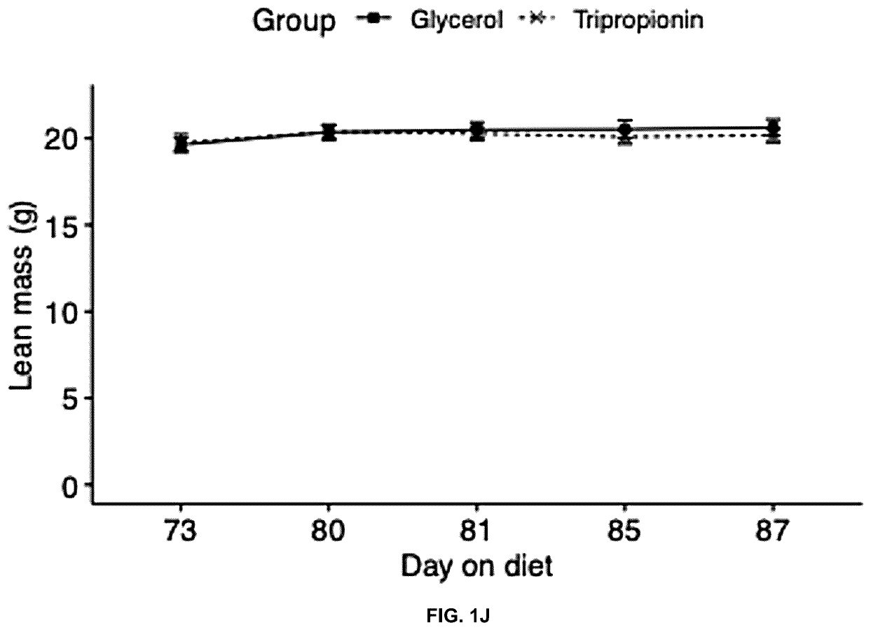

Tripropionin is a triglyceride containing three propionate fatty acid tails (FIG. 1A). Tripropionin lacks the odor associated with propionate salts and is therefore more palatable. We assessed the effect of tripropionin on adiposity and glucose homeostasis in mice challenged with a high fat diet.

### Results

To assess the effect of tripropionin on adiposity and glucose homeostasis, we fed 11-week-old male C57BL/6J mice a high-fat diet (45% kcal from fat) (HFD) supplemented with either 5.3% tripropionin (w/w) or 5.3% glycerol as a control. Mice were maintained on these diets for 13 weeks, during which food consumption and body weight were measured and an oral glucose tolerance test (OGTT) and insulin tolerance test (ITT) were performed (FIG. 1A). Additionally, body composition was monitored via NMR during the last 2 weeks before sacrifice.

We found that tripropionin-fed mice had significantly reduced body weight compared to glycerol fed mice after 3 weeks on diet (FIG. 1B). The differences in body weight were maintained throughout the remainder of the study (FIG. 1H). This was not explained by differences in food consumption, as there was no significant difference in consumption between the two diets (FIG. 1C). Moreover, the only statistically significant difference in food consumption was observed 8 days after dietary treatment but was higher in the tripropionin group. Combined with the fact that the tripropionin diet was more calorie-dense than the glycerol diet (4.7 kcal/g vs 4.6 kcal/g, respectively), this suggested that the lack of weight gain in tripropionin-fed mice was not due to reduced caloric intake. Importantly, the differences in body weight were primarily due to differences in fat mass between diets (FIG. 1I). There were no differences observed in lean mass between the dietary treatment groups (FIG. 1J). Tripropionin-fed mice had improved glucose tolerance compared to glycerol fed mice (FIGS. 1D and 1E). Additionally, tripropionin supplementation improved insulin sensitivity compared to glycerol (FIGS. 1F and 1G).

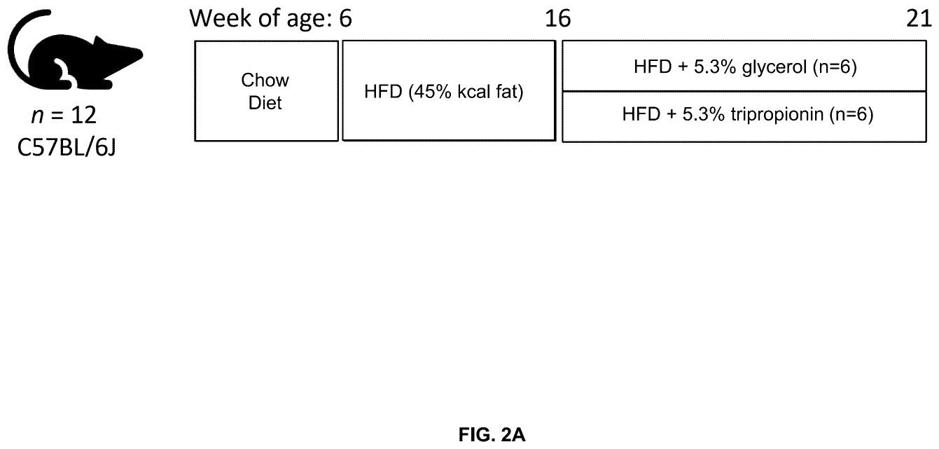

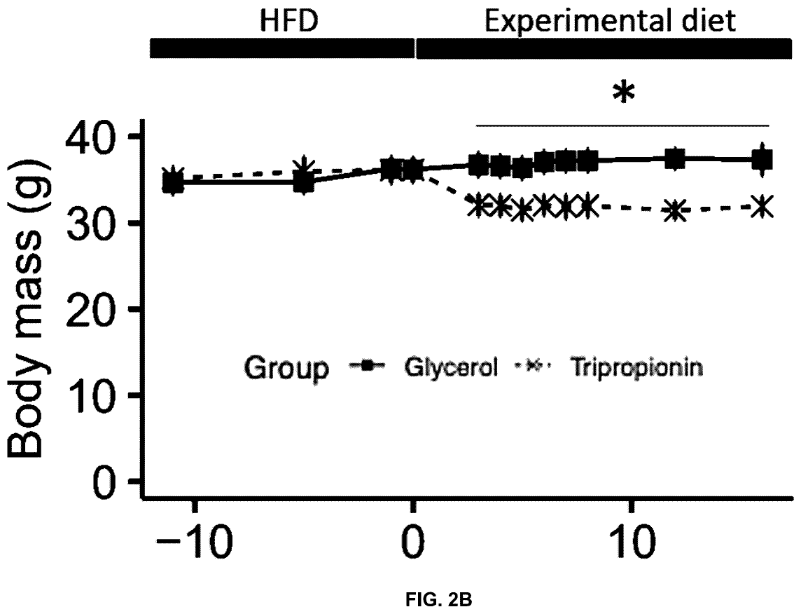

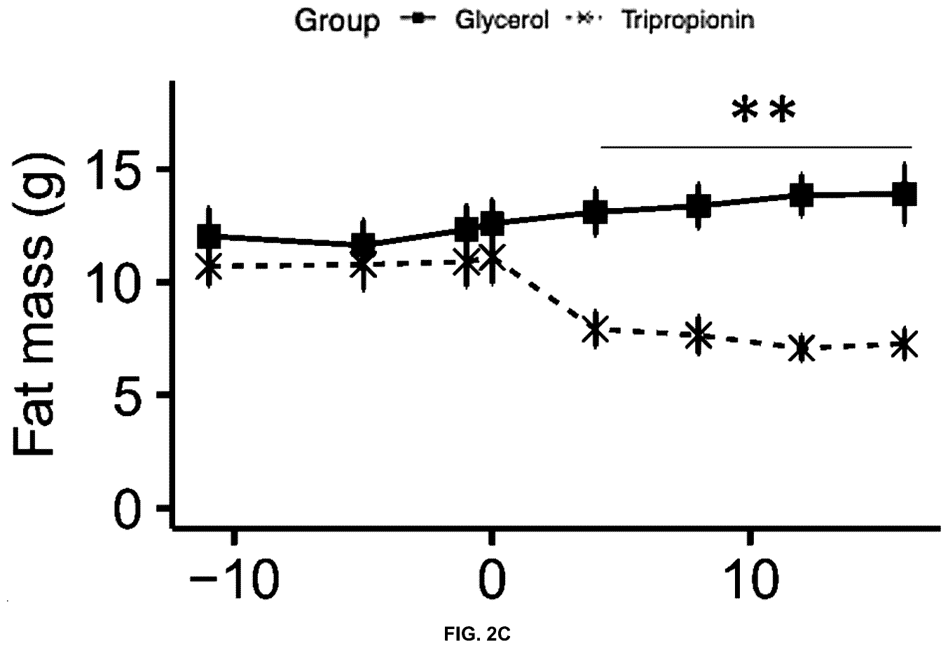

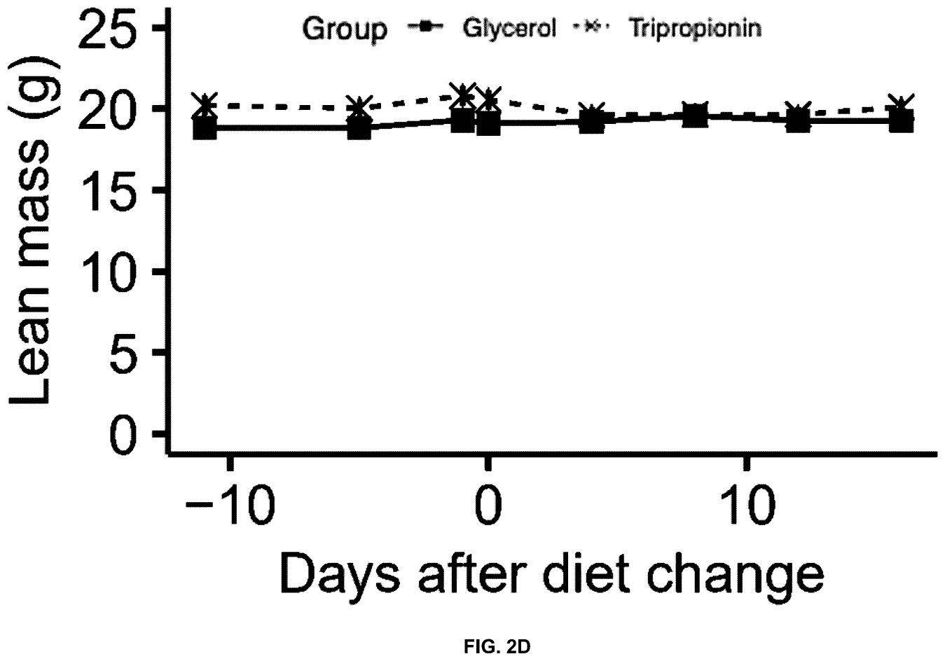

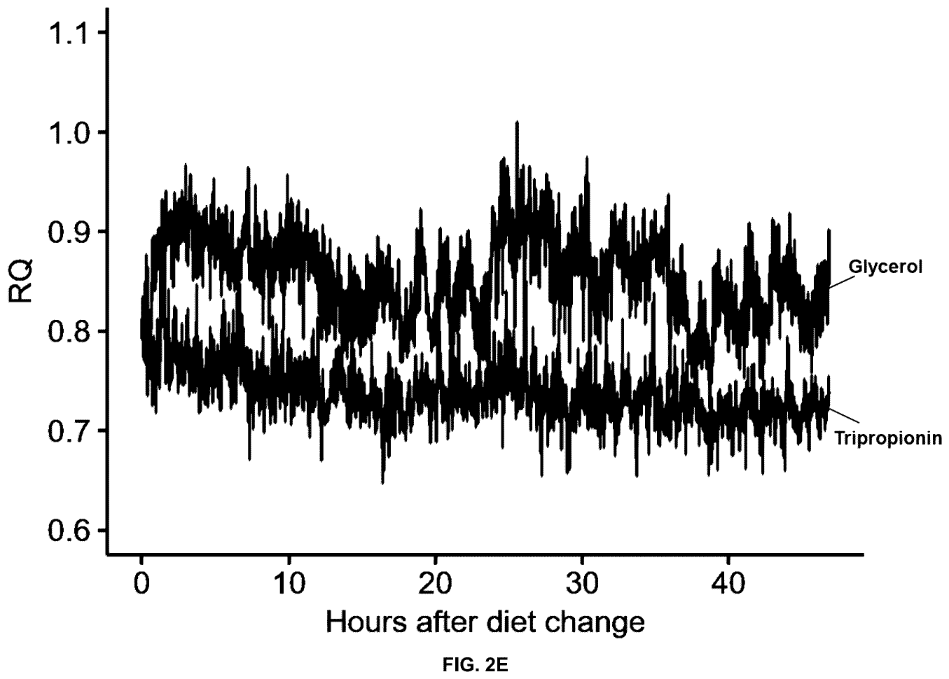

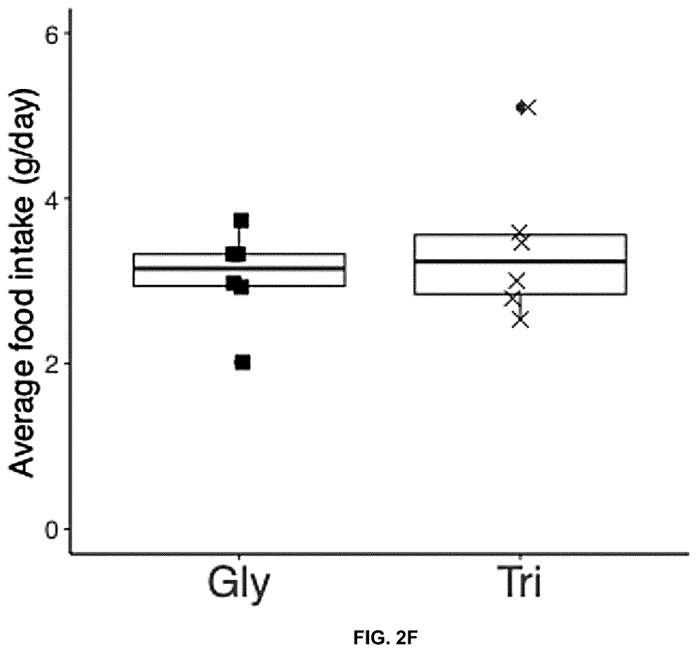

We next tested the anti-adipogenic effects of tripropionin in metabolically challenged mice. To do this, we fed a HFD for 10 weeks to induce insulin resistance and obesity and then switched mice to either the tripropionin or glycerol diet for 5 weeks (FIG. 2A). Consistent with the previous experiment, tripropionin-fed mice had reduced body weight compared to glycerol fed mice (FIG. 2B). Relative to the non-obese mice in the previous experiment, tripropionin led to a very acute reduction in body weight in obese mice with significant differences observed just three days after the diet change. As in the previous experiment, the reduction in body weight with tripropionin feeding was due entirely to a reduction in fat mass as there was no difference in lean mass between groups (FIGS. 2C and 2D). Mice were monitored in metabolic cages to measure respiratory quotient (RQ) for the initial 48 hours after diet change. The RQ of tripropionin-fed mice was reduced compared to the glycerol-fed mice immediately after diet change and this reduction was sustained for the duration of the 48-hour monitoring period (FIG. 2E).

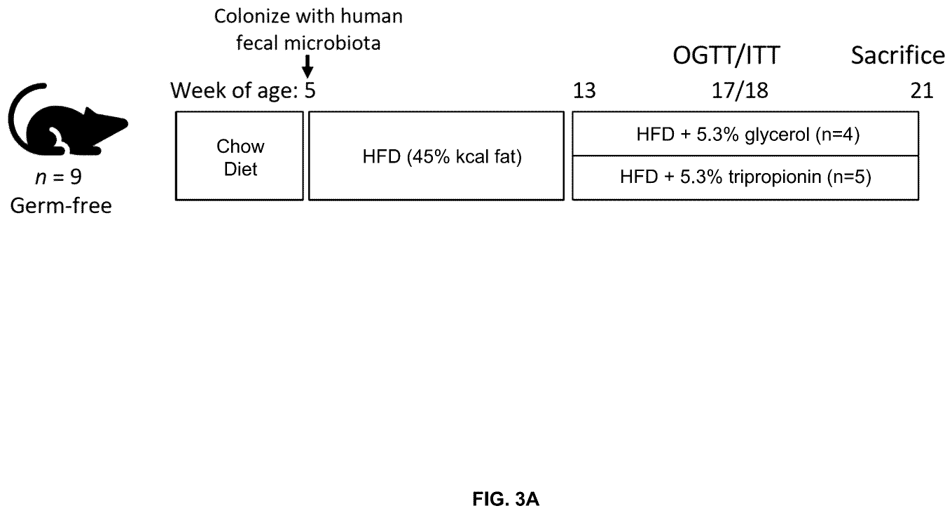

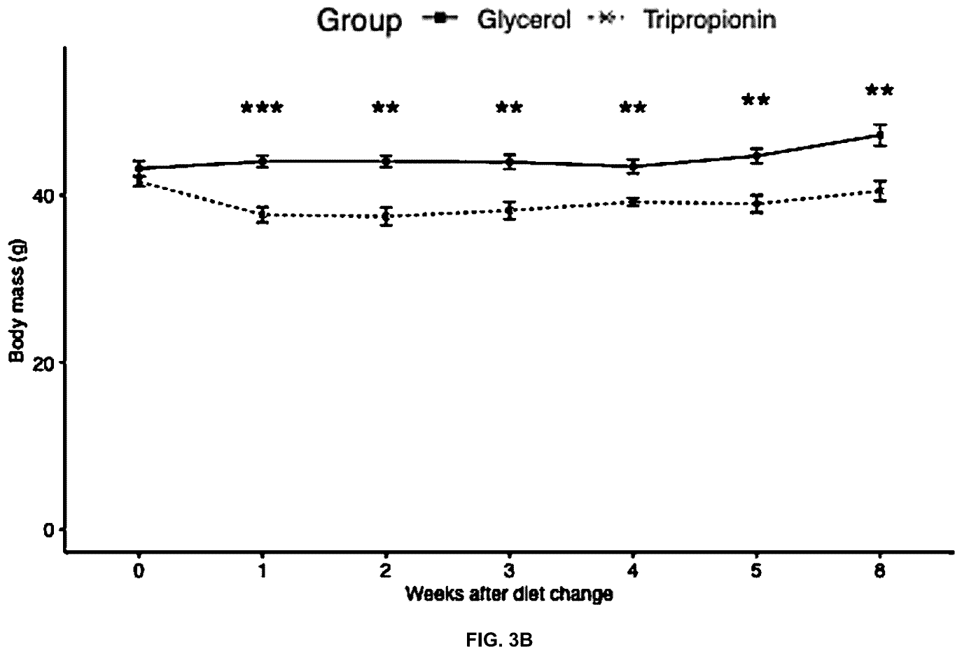

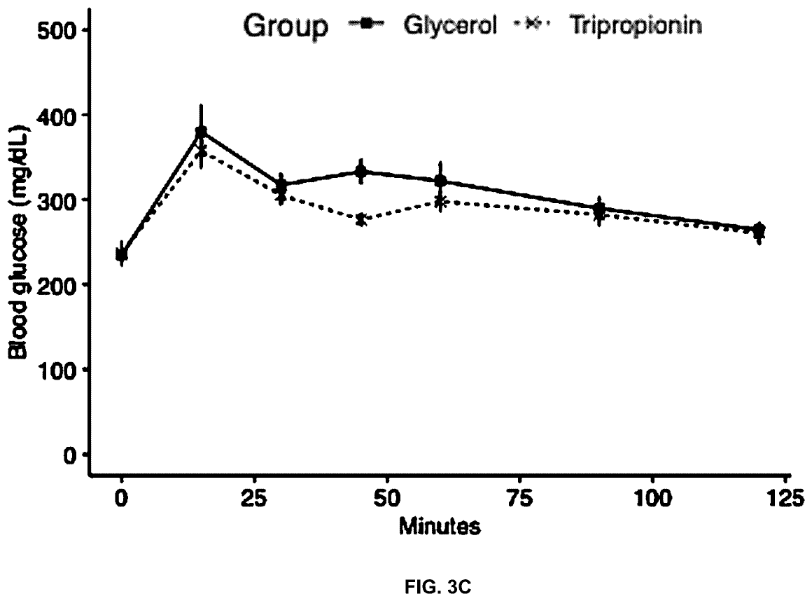

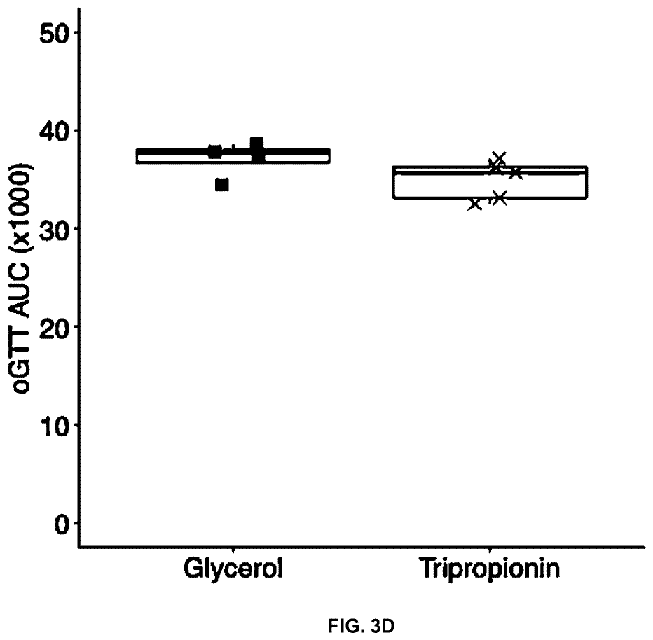

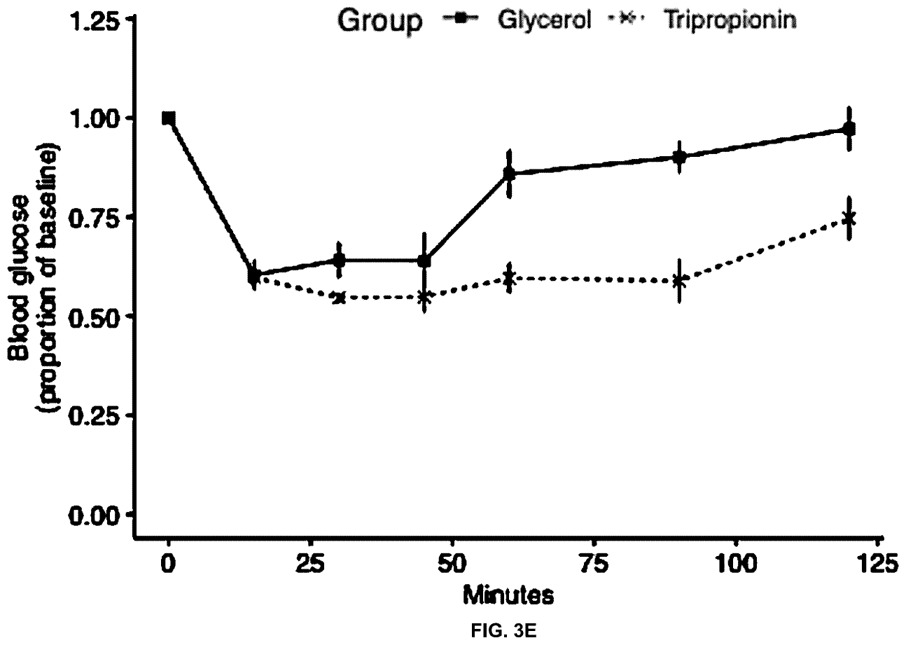

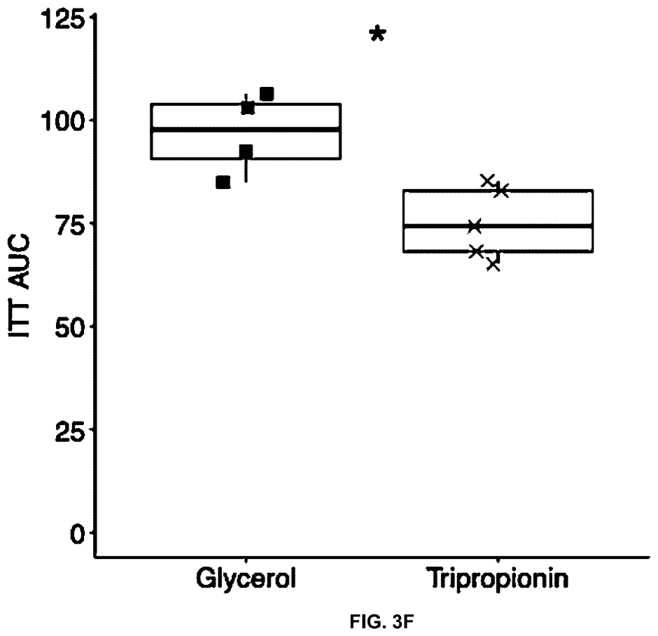

To check whether the effects of tripropionin supplementation are consistent in the context of a human microbiome, we used a gnotobiotic mouse model colonized with human fecal microbiota. We colonized germ-free mice with a complex community of human fecal microbiota, induced metabolic dysregulation though feeding a high-fat diet for 8 weeks, and subjected the mice to a tripropionin- or glycerol-supplemented diet (FIG. 3A). Consistent with the results above, tripropionin-fed mice had reduced body weight compared to glycerol fed mice (FIG. 3B). The tripropionin diet also improved insulin sensitivity compared to glycerol diet (FIGS. 3E and 3F) in the humanized mice.

Together these findings indicate that tripropionin protects against diet-induced adiposity and, in turn, improves glucose homeostasis. The effects of tripropionin on adiposity are consistent across conventionally raised mice and humanized gnotobiotic mice, but its effects on glucose tolerance seem to be more effective on non-obese mice. Tripropionin reduced RQ compared to glycerol-fed mice, indicating that tripropionin induces a metabolic shift that promotes fat oxidation. We suspect that, once ingested, intestinal lipases may liberate individual propionate molecules from tripropionin. This may deliver propionate over a greater distance and during a longer time period than oral administration of propionate salts, which appear to be rapidly absorbed in the proximal intestine.

### Materials and Methods

### Mouse Studies

Experiment 1: Male C57BL/6J mice were ordered from Jackson Laboratories (Bar Harbor, ME) maintained on chow (Teklad 8604, Inotiv, Madison, WI) and housed in a chlorinated water rack system in cages with cornhusk bedding and ALPHA-twist™. At 11 weeks of age, the mice were switched to either a glycerol (TD220541) or tripropionin diet (TD220540) from Envigo (Madison, WI) with a sample size of 6 mice per treatment. Four weeks after the diet change, mice were subjected to an OGTT. One week later, mice were subjected to an ITT. Mice were maintained on their respective diets for an additional 8 weeks and then transferred to single-housed metabolic cages (Promethion Core system, Sable Systems International, North Las Vegas, NV) where body weight food intake and activity level was monitored. During this period, fat and lean mass were monitored using nuclear magnetic resonance (NMR) machine fitted for mice (LF90 Body Composition Analyzer, Bruker Corporation, Billerica, MA). Mice were sacrificed at 24 weeks of age.

Experiment 2: Male C57BL/6J mice were ordered from Jackson Laboratories (Bar Harbor, ME) maintained on a chow diet and housed in a chlorinated water rack system. Mice were switched to the HFD and maintained on this diet for 10 weeks at which point mice were fed either the glycerol or the tripropionin diets (n=6 mice per group). Mice were transferred to singly housed metabolic cages, as described above, to measure respiratory quotient, body weight, food intake, and energy expenditure. Five weeks after diet change mice underwent an OGTT and then an ITT one week later.

Experiment 3: Germ-free (GF) male mice (C57BL/6J) were housed in sterile isolators and maintained on autoclaved chow (LabDiet 5021; LabDiet, St. Louis, MO) and sterile water ad libitum. GF cages contained Alpha-dri® (Shepherd Specialty Papers, Kalamazoo, MI) bedding along with paper huts (Bio-Huts, Bio-Serv, Flemington, NJ) and ALPHA-twist™ (Shepherd Specialty Papers) for enrichment. Monthly tests were conducted in each isolator to confirm GF status of the mice. These included a growth test of feces in rich media for 7 days at 37° C. and checking for amplification of the 16S rRNA gene using universal primers. At 5 and 6 weeks of age mice were placed in Allentown Sentry SPP IVC rack system (Allentown Inc., Allentown, NJ) and fed a HFD (TD08811, Envigo). Mice were then colonized with fecal microbiota from a human donor fecal specimen via gavage with a fecal slurry. The fecal slurry was prepared by collecting 200-500 mg of frozen feces and adding it to anaerobic CMM broth in an anaerobic chamber. The mixture was vortexed for 1 minute and then placed on ice until gavaging (less than 1 hour after preparation). Mice were maintained on the HFD for an additional 6 weeks before being switched to either the glycerol or tripropionin diets (n=4-5 per group). Four weeks after diet change mice were subjected to an OGTT followed by an ITT a week later. Mice were euthanized one week after ITT.

### Oral Glucose Tolerance Test

Prior to OGTT, mice were placed in fresh cages fasted for 4 hours. Baseline blood glucose measurements were taken using a glucometer from a drop of blood from the tail. After collecting fasting blood glucose measurements, mice were immediately gavaged with a bolus of 2 g of glucose (45%) per Kg of body weight. Blood glucose levels were measured at regular intervals between 15 and 120 minutes.

### Insulin Tolerance Test

The ITT was performed one week after the OGTT. Mice were fasted for 4 hours in fresh cages and baseline blood glucose levels were taken from tail blood. Insulin was administered at a rate of 0.75 IU per Kg body weight via IP injection. Blood glucose levels were monitored as described for OGTT. Insulin tolerance curves are expressed as the relative ratio of blood glucose from the baseline.

### Example 2

### Background

Propionate is a short-chain fatty acid produced by microbiota in the intestine, plasma levels of which have been associated with improved insulin sensitivity. There are conflicting reports as to whether supplementation with sodium propionate is beneficial or detrimental to the host. This discrepancy may be due to the fact that sodium propionate is absorbed in the proximal gut, whereas fiber-derived propionate is absorbed more distally. Tripropionin is a triglyceride made up of a glycerol backbone with three propionic acid tails and is acted upon by lipases to liberate free propionate molecules. In this way, tripropionin likely delivers propionate to more-distal regions of the intestine than sodium propionate, thereby better mimicking the action of fiber-derived propionate. We have found in Example 1 that tripropionin supplementation in a high-fat diet (5.3% w/w) has been shown to reduce adiposity and improve glycemic control in mice, but the effective minimal dose has not been determined.

### Objective

Determine the minimal effective dose of dietary tripropionin supplementation on glycemic control and adiposity in obese mice.

### Design

Narrative: 6 week old male and female mice will be fed a high-fat diet (HFD) (45% kcal from fat) diet for 8 weeks to induce insulin resistance and obesity. Mice will then be split in dietary treatment groups (see below) for 7 weeks and then euthanized. Four weeks after diet change, mice will be subjected to an OGTT. Six weeks after diet change, mice will be subjected to an ITT. Body mass and food consumption will be monitored throughout the duration of the study.

The mice will be split in the following groups: Control (−HFD); HFD+5% w/w glycerol; HFD+0.5% w/w tripropionin; HFD+2.5% w/w tripropionin; HFD+5% w/w tripropionin.

The HFD will be 45% kcal from fat. The experimental diets will add their respective component at the expense of starch, by weight.

Each group will have 8 mice, housed across two cages. The groups will be repeated for males and females. 80 mice total will be tested. The total duration of the experiment will be 15 weeks.

Baseline measures (prior to the start of the dietary treatment) will include fasting glucose, fasting insulin, and fat mass.

Endpoint measures will include OGTT/ITT and fat mass as primary outcomes. Endpoint measures will also include plasma and liver lipids (TAG, FFA, cholesterol), intestinal histology, GLP-1 levels, tissues collection, markers of inflammation in the intestine, and liver enzymes, as secondary outcomes.

### Example 3

### Objective

Determine whether a low dose of over-the-counter lipase inhibitor (orlistat, CAS Number 96829-58-2, N-formyl-L-leucine (1S)-1-[[(2S,3S)-3-hexyl-4-oxo-2-oxetanyl]methyl]dodecyl ester) potentiates the effects of tripropionin supplementation on glycemic control and adiposity in obese mice. We hypothesize that inhibition of lipases in the proximal gut with a low dose of orlistat (20 ppm) will delay the release of propionate from tripropionin, causing an increase in propionate levels more distally in the small intestine where higher concentration of GLP-1 releasing cells are detected, thus having a stronger GLP-1 response and associated metabolic benefits.

### Design

Narrative: 6 week old male and female mice will be fed a high-fat diet (HFD) (45% kcal from fat) diet for 8 weeks to induce insulin resistance and obesity. Mice will then be split in dietary treatment groups (see below) for 7 weeks and then euthanized. Four weeks after diet change, mice will be subjected to an OGTT. Six weeks after diet change, mice will be subjected to an ITT. Body mass and food consumption will be monitored throughout the duration of the study.

The mice will be split in the following groups: HFD+20 ppm orlistat (control); HFD+20 ppm orlistat+0.1% tripropionin; HFD+20 ppm orlistat+0.5% tripropionin.

The HFD will be 45% kcal from fat. The experimental diets will add their respective component at the expense of starch, by weight.

Each group will have 8 mice, housed across two cages. The groups will be repeated for males and females. 80 mice total will be tested. The total duration of the experiment will be 15 weeks. Size effect of combined therapy relative to control will be compared to those seen above with single tripropionin therapy.

Baseline measures (prior to the start of the dietary treatment) will include fasting glucose, fasting insulin, and fat mass.

Endpoint measures will include OGTT/ITT and fat mass as primary outcomes. Endpoint measures will also include plasma and liver lipids (TAG, FFA, cholesterol), intestinal histology, GLP-1 levels, tissues collection, markers of inflammation in the intestine, and liver enzymes, as secondary outcomes. We expect that the combination of therapy will have synergistic effects relative to tripropionin alone.

### REFERENCES

Chambers E S, Viardot A, Psichas A, Morrison D J, Murphy K G, Zac-Varghese S E, MacDougall K, Preston T, Tedford C, Finlayson G S, Blundell J E, Bell J D, Thomas E L, Mt-Isa S, Ashby D, Gibson G R, Kolida S, Dhillo W S, Bloom S R, Morley W, Clegg S, Frost G. Effects of targeted delivery of propionate to the human colon on appetite regulation, body weight maintenance and adiposity in overweight adults. Gut. 2015 November; 64 (11): 1744-54.
Ellis A C, Hyatt T C, Hunter G R, Gower B A. Respiratory quotient predicts fat mass gain in premenopausal women. Obesity (Silver Spring). 2010 December; 18 (12): 2255-9.
Fernandes J, Su W, Rahat-Rozenbloom S, Wolever T M, Comelli E M. Adiposity, gut microbiota and faecal short chain fatty acids are linked in adult humans. Nutr Diabetes. 2014 Jun. 30; 4 (6):e121).
Müller M, Hernández M A G, Goossens G H, Reijnders D, Holst J J, Jocken J W E, van Eijk H, Canfora E E, Blaak E E. Circulating but not faecal short-chain fatty acids are related to insulin sensitivity, lipolysis and GLP-1 concentrations in humans. Sci Rep. 2019 Aug. 29; 9(1): 12515.
Tirosh A, Calay E S, Tuncman G, Claiborn K C, Inouye K E, Eguchi K, Alcala M, Rathaus M, Hollander K S, Ron I, Livne R, Heianza Y, Qi L, Shai I, Garg R, Hotamisligil G S. The short-chain fatty acid propionate increases glucagon and FABP4 production, impairing insulin action in mice and humans. Sci Transl Med. 2019 Apr. 24; 11(489):eaav0120.
Widmaier, Eric P.; Raff. Hershel; Strang. Kevin T. (2016). Vander's Human Physiology: The Mechanisms of Body Function (14th ed.). New York: McGraw Hill. ISBN 9781259294099.

The invention claimed is:

1. A method of reducing adiposity and/or improving glucose homeostasis in a subject, the method comprising orally administering an effective amount of isolated tripropionin to the subject.

2. The method of claim 1, wherein the tripropionin is administered in an amount effective to:
reduce body weight in the subject;
reduce fat mass in the subject;
improve glucose tolerance in the subject;
improve insulin sensitivity in the subject; and/or
reduce respiratory quotient of the subject.

3-7. (canceled)

8. The method of claim 1, wherein the subject, when the administering initially commences, has a body mass index greater than 25.

9. The method of claim 1, wherein the subject is a male who, when the administering initially commences, has a body fat percentage greater than 15%, or the subject is a female who, when the administering initially commences, has a body fat percentage greater than 25%.

10. The method of claim 1, wherein the subject, when the administering initially commences, has a fasting glucose greater than 100 mg/dL.

11. The method of claim 1, wherein the subject, when the administering initially commences, has a condition selected from the group consisting of obesity, hyperglycemia, prediabetes, diabetes (e.g., type 1 diabetes type 2 diabetes, gestational diabetes, and maturity-onset diabetes of the young), insulin resistance, metabolic syndrome, Alzheimer's disease, atherosclerosis, and fatty liver disease, and wherein the tripropionin is administered in an amount effective to treat the condition.

12. The method of claim 1, further comprising administering a lipase inhibitor to the subject.

13. The method of claim 1, wherein the tripropionin is administered in a composition comprising the tripropionin and a carrier.

14. The method of claim 13, wherein the composition is a solid or semisolid.

15. The method of claim 13, wherein the composition is in the form a pill.

16. The method of claim 15, wherein the pill comprises the tripropionin in an amount of at least 25 mg.

17. The method of claim 13, wherein the composition is a liquid.

18. (canceled)

19. The method of claim 13, wherein the composition comprises the tripropionin in an amount of at least 5% w/w.

20. The method of claim 13, wherein the composition has a caloric density no greater than 0.15 kcal/g.

21. The method of claim 13, wherein the composition has a caloric density of at least 0.4 kcal/g.

22. The method of claim 21, wherein at least 5% of total calories in the composition is protein.

23. The method of claim 21, wherein at least 5% of total calories in the composition is digestible carbohydrate.

24. The method of claim 21, wherein at least 5% of total calories in the composition is fat.

25. The method of claim 13, wherein the composition further comprises a lipase inhibitor.

26. A composition as recited in claim 13.
